(19) United States
(12) Patent Application Publication
(10) Pub. No.: US 2024/0350531 A1
(43) Pub. Date: Oct. 24, 2024

(54) FUCAN AND MODIFIED FUCAN COMPOSITIONS FOR THE TREATMENT OF CONDITIONS RELATED TO CAPSULAR CONTRACTURE AND TO INHIBITING FIBROUS GROWTH AROUND OR ON TRANSPLANTS

(71) Applicant: ARC Medical Inc., Richmond (CA)

(72) Inventors: Hesong Sun, Richmond (CA); Ian Millet, Richmond (CA); Christopher Michael Kevin Springate, Richmond (CA)

(73) Assignee: ARC Medical Inc., Richmond, BC (CA)

(21) Appl. No.: 18/684,909

(22) PCT Filed: Aug. 18, 2022
(86) PCT No.: PCT/CA2022/051254

§ 371 Date: Feb. 20, 2024

Related U.S. Application Data
(60) Provisional application No. 63/235,316, filed on Aug. 20, 2021.
(60) Provisional application No. 63/354,322, filed on Jun. 22, 2022.

(51) Int. Cl. A61K 31/737; A61P 19/04; A61P 37/06

(52) U.S. Cl. CPC A61K 31/737; A61P 19/04; A61P 37/06

(57) ABSTRACT

Compositions and methods comprising medically-acceptable fucans suitable for medical and surgical applications, including treatment of capsular contracture and other foreign body reaction (FBR) conditions, and medical and surgical applications related to transplants and transplant operations, such as GVHD and fibrous growth around or on implants or transplants after implantation/transplantation, and related diseases, infections, and traumas.

### BACKGROUND

Fucans (including fucoidan) are sulfated polysaccharides. In general terms, this means that they are molecules made up of a number of sugar groups, and also have sulfur atoms attached to the sugar groups. The main sugar group is called “fucose”, which is sugar that has 6 carbon atoms and has the chemical formula C6H1205. “Fucoidan” (or fucoidin) indicates fucans derived from brown algae (seaweed). Fucans can exist alone, or in a mixture of other sugars, for example in a mixture of sugars such as xylose, galactose, glucose, glucuronic acid and/or mannose. These other sugars may be extracted from the seaweed or other source with the fucan. Although fucans are currently derived from natural sources such as the brown algae (seaweeds), sea cucumbers, etc., mentioned herein, “fucan” includes polymer molecules having the chemical and structural motifs of the fucans as discussed herein regardless of the ultimate source(s) of the fucans.

Fucoidan can be obtained from a variety of species of brown algae including but not limited to: Adenocystis utricularis, Ascophyllum nodosum, Chorda filum, Cystoseirabies marina, Durvillaea antarctica, Ecklonia kurome, Ecklonia maxima, Eisenia bicyclis, Fucus evanescens, Fucus vesiculosis, Hizikia fusiforme, Himanthalia Elongata, Kjellmaniella crassifolia, Laminaria brasiliensis, Laminaria cichorioides, Laminaria hyperborea, Laminaria japonica, Laminaria saccharina, Lessonia trabeculata, Macrocystis pyrifera, Pelvetia fastigiata, Pelvetia Canaliculata, Saccharina japonica, Saccharina latissima, Sargassum stenophylum, Sargassum thunbergii, Sargassum confusum, Sargassum fusiforme and Undaria pinnatifida. These exemplary species are all from the taxonomic class Phaeophyceae and the majority of these species fall into the families of Fucales and Laminariaceae.

As used herein, “transplant” means a surgical operation in which an organ or tissue is transplanted into or onto a body, namely the surgical insertion of an organ or tissue from one body to another, typically within species, or from one location within a body to another location within the same body. The “transplant” is an organic whole organ or tissue and thus is distinguished from implants, and the like, such as a replacement knee or replacement ocular lens, which implant is typically a non-organic item although implants can include organically derived components such as biologic elements derived from blood, vaccines, allergenics, tissues, cells, and cellular and gene therapies.

The current disclosure provides compositions and methods comprising fucans for the treatment of capsular contracture or conditions related to transplants and transplant operations, such as graft-versus-host-disease (GVHD, https://en.wikipedia.org/wiki/Graft-versus-host disease) and to inhibiting fibrous growth on or around implants or transplants, for which there has gone an unmet need for compounds, compositions, methods and the like (including delivery approaches) to treat these capsular contractures, transplant or implant conditions, including providing a treatment with few side effects. The present systems and methods, etc., provide these and/or other advantages.

### INCORPORATION BY REFERENCE

The present application incorporates by reference in their entirety the following publications: PCT/CA2019/051025, filed Jul. 24, 2019; PCT/CA2019/051026, filed Jul. 24, 2019; PCT/CA2019/051027, filed Jul. 24, 2019, PCT/CA2019/051030, filed Jul. 24, 2019; PCT/CA2019/051028, filed Jul. 24, 2019; PCT/CA2020/050294, filed Mar. 5, 2020; PCT/CA2020/050295, filed Mar. 5, 2020; and PCT/CA2019/051029, filed Jul. 24, 2019.

### SUMMARY

As noted above, the current application is directed to compositions and methods comprising medically-acceptable fucans suitable for medical and surgical applications, including the treatment of capsular contracture and other foreign body reaction (FBR) conditions. The current application is also directed to compositions and methods comprising medically-acceptable fucans suitable for medical and surgical applications related to transplants and transplant operations, such as GVHD and fibrous growth around or on implants or transplants after implantation/transplantation, and related diseases, infections, and traumas. Collectively such transplant-related GVHD, fibrous growth problems, etc., shall be referred to herein as “transplant conditions.” The present compositions and methods include medically-acceptable fucans effective to treat issues such as FBR and transplant conditions in therapeutically effective medical compositions. In some embodiments, the medically-acceptable fucan is fucoidan. The present medically-acceptable fucans can themselves be, or can be included on or in medical compositions including for example medically acceptable medical devices, biologics (biologics herein includes products that are derived from living sources such as animals including humans, and microorganisms, for example blood, vaccines, allergenics, tissues, cells, and cellular and gene therapies), drugs, combination products, pharmaceutical compositions, biopharmaceuticals and other medically acceptable, therapeutically and/or medically effective compositions, all of which are collectively referred to as “medical compositions” herein.

In some embodiments, Patients receive a medical composition comprising a medically-acceptable fucan as discussed herein to treat a capsular contracture, fibrous growth around implants, etc. as discussed herein. In some embodiments, Patients receive transplant condition medical compositions comprising a medically-acceptable fucan as discussed herein to treat transplant conditions. Treatment herein includes the inhibition, prevention, removal, reduction or other treatment of the capsular contracture or fibrous growth around implants discussed herein. The medical compositions discussed herein can comprise medically-acceptable fucans with a suitably low endotoxin level for use in a medical and/or surgical setting. The compositions herein may further comprise medically-acceptable fucans having a desired molecular weight distribution and/or sulfate level. The treatment can comprise administering the medical composition at a suspected site, wherein suspected indicates that a practitioner believes the site is already affected by the capsular contracture or fibrous growth around foreign bodies and/or transplants or is at a heightened risk compared to pre-implant/transplant risk levels of contracting the capsular contracture or fibrous growth around implants or transplant conditions.

The present systems, devices and methods, etc., provide methods of treating certain capsular contracture or fibrous growth or transplant conditions around transplants, for example signal activity such as cytokine or chemokine activity is a component of the condition. In some embodiments, the treating comprises inhibiting the presence or formation of such capsular contracture or fibrous growth around implants/transplants at a particular site in a patient. For example, the methods comprise administering a therapeutically effective amount of a medically acceptable fucan composition to the site of the capsular contracture or fibrous growth around implants/transplants in the patient suspected of having the capsular contracture or fibrous growth around implants or transplants.

In some embodiments, the methods further comprise, before administering the medically acceptable fucan composition to the patient, identifying a need for the inhibiting of capsular contracture or fibrous growth around implants in the patient and then selecting the medically acceptable fucan composition specifically for administering to the patient to effect the inhibiting or other treating. In some embodiments, the administering, treating and/or inhibiting includes rinsing a specific treatment site suspected of having the capsular contracture or fibrous growth around implants, or otherwise directly administering the medically acceptable fucan composition to the site having or suspected of having the capsular contracture or fibrous growth around implants. Rinsing the target area with compositions containing the fucan compositions herein as the administration and/or treatment/inhibition modality can be particularly advantageous.

In some aspects of the present systems, devices and methods, etc., the administering comprises directly delivering the medically acceptable fucan composition to the site having or suspected of having capsular contracture or fibrous growth around implants, for example via an instillate, rinse, gel or powder; such does not include systemic administration such as via an oral medication. The medically acceptable fucan composition can be substantially continuously administered to the site having or suspected of having capsular contracture or fibrous growth around implants via controlled release from a polymeric or other acceptable controlled release dosage form. The medically acceptable fucan composition can be administered intravenously, intraarticularly, intralesionally, intravaginally, rectally, intramuscularly, intraperitoneally, subcutaneously, topically, intranasally, intraocularly or orally.

In further aspects, the present systems, devices and methods, etc., provide methods of treating capsular contracture or fibrous growth around implants in a patient, comprising coating at least one surface of an implant with a therapeutically effective amount of a medically acceptable fucan composition before delivering the implant to the patient. Such methods can further comprise, before coating the surface of the implant with the medically acceptable fucan composition, identifying a need for the treating the capsular contracture or fibrous growth around implants in the patient and then selecting the medically acceptable fucan composition for the coating of the surface of the implant with the medically acceptable fucan composition to effect the treating.

The medically acceptable fucan compositions can further comprise at least one medically acceptable excipient selected from the group consisting of gelatin, hypromellose, lactose, water for injection USP, sodium chloride, sodium phosphate, sodium citrate, sodium ascorbate, phosphate buffers, citrate buffers, phosphate-citrate buffers, pluronic, cellulose, alginate, acrylate, hyaluronic acid, polyethylene glycol, poly(lactic acid), poly(lactic-co-glycolic acid), carrageenan, polyurethane, polyethylene, polypropylene, polytetrafluoroethylene, chitosan and lactated Ringer's injection USP. The patient can be an animal including in some instances a human. The medically acceptable fucan composition can comprise fucoidan, and the embodiments, etc., herein include use of a medically acceptable fucan composition to treat, including inhibit, the capsular contracture or fibrous growth around implants as discussed herein. Also included herein are compositions comprising a medically acceptable modified fucan for treating, including inhibiting, at least one of the capsular contracture or fibrous growth around implants herein including, e.g., capsular contracture; foreign body response; fibrous capsule formation; and biofilm infections.

In some embodiments, the compositions comprise a medically acceptable modified fucan with less than about 0.2 EU/mg, 0.1 EU/mg, 0.05 EU/mg, 0.02 EU/mg, 0.01 EU/mg, 0.005 EU/mg, 0.002 EU/mg. In further embodiments, the compositions can comprise less than about 0.40% w/w total nitrogen, less than about 0.20% w/w total nitrogen, less than about 0.15% w/w total nitrogen or less than about 0.10% w/w total nitrogen. The modified medically acceptable fucan can comprise between about 10% w/w and 60% w/w sulfate or between about 30% w/w and 60% w/w sulfate. The modified medically acceptable fucan can comprise a molar ratio of total sulfate to total fucose of between 0.5 and 3.0.

The modified medically acceptable fucan can comprise a molar ratio of total sulfate to total fucose of between 1.1 and 3.0. The modified medically acceptable fucan can comprise a molar ratio of total sulfate to total fucose and galactose of between about 0.5 and 3.0, or of between about 1.1 and 3.0. The modified medically acceptable fucan can comprise a total carbohydrate content of between about 27% w/w and 80% w/w, or between about 30% w/w and 70% w/w. The modified medically acceptable fucan can comprise a fucose content as a percentage of the total carbohydrate of more than about 30% w/w, 40% w/w, 50% w/w, 70% w/w, 80% w/w, or 90% w/w. The modified medically acceptable fucan can comprise a galactose content as a percentage of the total carbohydrate of less than about 60% w/w, 30% w/w, 20% w/w, or 10% w/w. The modified medically acceptable fucan can comprise a total glucuronic acid, mannose, rhamnose and xylose content as a percentage of the total carbohydrate content of less than about 30% w/w, 20% w/w, 10% w/w or 5% w/w.

The present systems, devices and methods, etc., herein also provide:

Methods of treating a fibrous capsule formation in a patient at a site of an implant on or within the patient, the methods can comprise treating the fibrous capsule formation with a therapeutically effective amount of a medically acceptable fucan compositions. In some embodiments.

Methods of treating a foreign body response in a patient at a site of an implant on or within the patient, the methods can comprise treating the foreign body response with a therapeutically effective amount of a medically acceptable fucan compositions.

Methods of treating a capsular contracture in a patient at a site of an implant on or within the patient, the methods can comprise treating the capsular contracture with a therapeutically effective amount of a medically acceptable fucan compositions. Methods of treating a biofilm infection in a patient at a site of an implant on or within the patient, the methods can comprise treating the biofilm formation with a therapeutically effective amount of a medically acceptable fucan compositions.

The treating can comprise inhibiting, and the implant can comprise at least one of a medical device, drug or combination product. The implant can be composed of at least one of a non-synthetic, biologic, naturally derived and synthetic material. The implant can comprise at least one of a bone plate, fracture fixation device, hip prostheses, knee prostheses, shoulder prostheses, ankle prostheses, elbow prostheses, artificial ligament, artificial tendon, cellular therapy, gene therapy, pacemaker encapsulation, catheter, stent, artificial heart valve, artificial artery, drug reservoir device for sustained release, diabetes monitor, insulin pump, skin repair device, breast implant, cochlear replacement, ocular lens, vascular graft, nerve conduit, surgical mesh, organ, tissue and cell. The implant can be composed of at least one of: autograft, allograft, fibrin, poly(lactic acid), poly(lactic-co-glycolic acid), alginate, carrageenan, hyaluronan, heparin, synthetic polyurethane, polyester, silicone, aluminium, steel, titanium, cobalt, chromium, nickel, gold, silver, platinum, metal alloy, calcium phosphate, hydroxyapatite, inorganic salt derivatives, alumina, zirconia, bioactive glass, porcelain, carbon, cyclic olefin copolymer, polycarbonate, polyetherimide, polyvinylchloride, polyethersulfone, polyethylene, polytetrafluoroethylene, polyetheretherketone, polypropylene, silicone, hydrogel, cellulose, starch, protein, peptide, DNA, RNA, collagen, gelatin, silk, chitin, chitosan, glucose, heart valve, blood vessel and liver tissue.

The treating with the therapeutically effective amount of the medically acceptable fucan compositions can comprise coating at least one surface of the implant with the therapeutically effective amount of the medically acceptable fucan compositions before delivering the implant to the patient. The treating with the therapeutically effective amount of the medically acceptable fucan compositions can comprise coating at least one surface of the implant with the therapeutically effective amount of the medically acceptable fucan compositions after delivering the implant to the patient. The treating with the therapeutically effective amount of the medically acceptable fucan compositions can comprise embedding the therapeutically effective amount of the medically acceptable fucan compositions within the implant before delivering the implant to the patient.

The treating with the therapeutically effective amount of the medically acceptable fucan compositions can comprise embedding the therapeutically effective amount of the medically acceptable fucan compositions within the implant after delivering the implant to the patient. The treating with the therapeutically effective amount of the medically acceptable fucan compositions can comprise co-administering the therapeutically effective amount of the medically acceptable fucan compositions with the implant. The treating with the therapeutically effective amount of the medically acceptable fucan compositions can comprise administering the therapeutically effective amount of the medically acceptable fucan compositions before delivering the implant to the patient. The treating with the therapeutically effective amount of the medically acceptable fucan compositions can comprise administering the therapeutically effective amount of the medically acceptable fucan compositions after delivering the implant to the patient.

The administering can occur at the site of the implant, and the patient can be an animal.

The medically acceptable fucan compositions can comprise at least one of a paste, gel, patch, film, spray, liquid, lotion, cream, solution, suspension, solid, implant, powder and microsphere. The medically acceptable fucan compositions further can comprise at least one medically acceptable excipient selected from the group consisting of gelatin, hypromellose, lactose, water for injection USP, sodium chloride, sodium phosphate, sodium citrate, sodium ascorbate, phosphate buffers, citrate buffers, phosphate-citrate buffers, pluronic, cellulose, alginate, acrylate, hyaluronic acid, polyethylene glycol, poly(lactic acid), poly(lactic-co-glycolic acid), alginate, carrageenan, polyurethane, polyethylene, polypropylene, polytetrafluoroethylene, chitosan and lactated Ringer's injection USP. The medically acceptable fucan compositions can comprise less than about 200 Endotoxin Units (EU), 100 Endotoxin Units (EU), 50 Endotoxin Units (EU), 20 Endotoxin Units (EU), 10 Endotoxin Units (EU), 5 Endotoxin Units (EU) or 2 Endotoxin Units (EU).

The methods, etc., herein include use of a medically acceptable fucan compositions to treat fibrous capsule formation, foreign body response, treat capsular contracture, biofilm infection, implant conditions, or transplant conditions.

The compositions can comprise an implant or transplant and a medically acceptable fucan composition. The implant can comprise at least one of a medical device, drug and combination product, and can be composed of at least one of a non-synthetic, biologic, naturally derived and synthetic material. The implant can comprise a bone plate, fracture fixation device, hip prostheses, knee prostheses, shoulder prostheses, ankle prostheses, elbow prostheses, artificial ligament, artificial tendon, cellular therapy, gene therapy, pacemaker encapsulation, catheter, stent, artificial heart valve, artificial artery, drug reservoir device for sustained release, diabetes monitor, insulin pump, skin repair device, breast implant, cochlear replacement, ocular lens, vascular graft, nerve conduit, surgical mesh, organ, tissue and cell. The implant can be composed of at least one of: autograft, allograft, fibrin, poly(lactic acid), poly(lactic-co-glycolic acid), alginate, carrageenan, hyaluronan, heparin, synthetic polyurethane, polyester, silicone, aluminium, steel, titanium, cobalt, chromium, nickel, gold, silver, platinum, metal alloy, calcium phosphate, hydroxyapatite, inorganic salt derivatives, alumina, zirconia, bioactive glass, porcelain, carbon, cyclic olefin copolymer, polycarbonate, polyetherimide, polyvinylchloride, polyethersulfone, polyethylene, polytetrafluoroethylene, polyetheretherketone, polypropylene, silicone, hydrogel, cellulose, starch, protein, peptide, DNA, RNA, collagen, gelatin, silk, chitin, chitosan, glucose, heart valve, blood vessel and liver tissue.

In some aspects, the methods comprise treating a transplant condition in a patient at a site of a transplant on or within the patient, the methods can comprise treating the transplant condition with a therapeutically effective amount of a medically acceptable fucan compositions. The treating can comprise inhibiting the transplant condition. The transplant can comprise at least one of a heart, kidney, liver, lung, pancreas, intestine, thymus, uterus, bone and tendon, corneae, skin, heart valve, nerve or vein transplant.

The treating with the therapeutically effective amount of the medically acceptable fucan compositions can comprise coating at least one surface of the transplant with the therapeutically effective amount of the medically acceptable fucan compositions before, during or after delivering the transplant to the patient. The treating with the therapeutically effective amount of the medically acceptable fucan compositions can comprise embedding the therapeutically effective amount of the medically acceptable fucan compositions within the transplant before, during or after delivering the transplant to the patient. The treating with the therapeutically effective amount of the medically acceptable fucan compositions can comprise co-administering the therapeutically effective amount of the medically acceptable fucan compositions with the transplant, or administering before or after delivering the transplant to the patient.

The administering can occur at the site of the transplant, and the patient can be an animal. The medically acceptable fucan compositions can comprise at least one of a paste, gel, patch, film, spray, liquid, lotion, cream, solution, suspension, solid, transplant, powder and microsphere. The medically acceptable fucan compositions further can comprise at least one medically acceptable excipient selected from the group consisting of gelatin, hypromellose, lactose, water for injection USP, sodium chloride, sodium phosphate, sodium citrate, sodium ascorbate, phosphate buffers, citrate buffers, phosphate-citrate buffers, pluronic, cellulose, alginate, acrylate, hyaluronic acid, polyethylene glycol, poly(lactic acid), poly(lactic-co-glycolic acid), alginate, carrageenan, polyurethane, polyethylene, polypropylene, polytetrafluoroethylene, chitosan and lactated Ringer's injection USP.

The methods, etc., herein include use of a medically acceptable fucan compositions to treat transplant condition. Compositions herein can comprise a transplant and a medically acceptable fucan composition adequate to treat a transplant condition.

These and other aspects, features and embodiments are set forth within this application, including the following Detailed Description. Unless expressly stated otherwise, all embodiments, aspects, features, etc., can be mixed and matched, combined and permuted in any desired manner.

### DETAILED DESCRIPTION

The current compositions, systems, methods, etc., discussed herein comprise medically-acceptable fucans configured to be effective for medical treatments, which can be, for example, during surgery or post-surgical. In some embodiments, signaling protein activity contributes to the initiation, progression, severity, prognosis etc., of such capsular contracture or fibrous growth around implants or transplants. In some embodiments, such treatments comprise the medically-acceptable fucans inhibiting or sequestering such signaling proteins. Signaling proteins include proteins such as cytokines and chemokines.

In some embodiments, the medically-acceptable fucan is fucoidan. The present medically-acceptable fucans can themselves be, or can be included on or in, medical devices, combination products, biologics or on or in pharmaceutically or medically acceptable, therapeutically and/or medically effective compositions. The compositions discussed herein can comprise medically-acceptable modified fucans with a desired, specific low endotoxin level for use in a medical and/or surgical setting. The compositions herein may further comprise medically-acceptable modified fucans having a desired, specific molecular weight distribution and/or sulfate level.

The following paragraphs turn to a brief discussion of some of the capsular contracture or fibrous growth around implants or transplants that can be treated with the medically-acceptable fucans discussed herein.

### Foreign Body Response and Fibrous Capsule Formation

The implantation or introduction of materials or implants, including biomaterials, medical devices, prosthesis tissue-engineered constructs and/or combination products, at a target site, for example, a surgical cavity, can result in the development of an inflammatory/fibrotic healing process response known as a foreign body reaction (FBR). The end stage of this healing process can result in fibrous encapsulation, where fibroblasts create a fibrous capsule, that may be vascularized and/or collagenous, that prevents the implanted material from interacting with surrounding tissue or reduces the ability of the implanted material to interact with surrounding tissue. The fibroblasts participating in the inflammation respond to signaling proteins including TGFb1, IL-1b, IL-6, IL-13, IL-33, prostaglandins, and leukotrienes at the site of the foreign material in the body.

Implants that are capable of causing FBR and fibrous capsule formation include without limitation: orthopedic implant devices such as bone plates, fracture fixation devices, hip, knee, shoulder, ankle and elbow joint prostheses, artificial ligaments and tendons, cardiovascular implants such as pacemaker encapsulation, defibrillators, catheters, stents, artificial heart valves and arteries, drug delivery systems such as drug reservoir devices for sustained release, monitoring devices such as diabetes monitors, insulin pumps, artificial tissues such as skin repair devices, breast implants, cochlear replacements, ocular lenses, vascular grafts, cerebral spinal fluid (CSF) shunt systems, permanent birth control, tissue-engineered constructs, nerve conduits, surgical meshes and organs, tissues and cells. Such implants can be non-synthetic, biological (derived from an animal such as a human), naturally derived or synthetic or a combination of non-synthetic, synthetic, naturally derived and biological materials. Examples of materials that can be used to make implants include without limitation: autografts, allografts, organic polymers, such as natural collagen, fibrin, chitosan, poly(lactic acid), poly(lactic-co-glycolic acid), alginate, carrageenan, hyaluronan, heparin, cellulose, and synthetic polyurethane (PU), polyester, silicone, metal, such as aluminium, steel, titanium, cobalt, chromium, nickel, gold, silver, platinum and alloys thereof, inorganic salts, such as calcium phosphate, hydroxyapatite, and their compounds or derivatives, ceramics such as alumina, zirconia, bioactive glass, porcelain, carbons, biocompatible plastics and polymers such as cyclic olefin copolymer, polycarbonate, polyetherimide, medical grade polyvinylchloride, polyethersulfone, polyethylene, polyetheretherketone, polytetrafluoroethylene, and polypropylene, silicones, hydrogels, biopolymers such as cellulose, starch, proteins, peptides, DNA, RNA, protein based biomaterials such as collagen, gelatin, silk, polysaccharide based biomaterials such as chitin, chitosan, glucose, decellularized tissue derived biomaterials such as heart valves, blood vessels, liver tissue, and other composites and/or combinations of these materials.

There are a lack of compositions capable of producing therapeutic effects at the implant site or surgical cavity. The compositions herein can be used to treat FBR and fibrous capsule formation and/or provide a significant therapeutic effect in patients suffering from these conditions. The compositions herein can be used to coat the surface of the implant, be co-administered with or after the implant, or be embedded within the implant. The compositions herein can be configured to release over time (delayed release) to achieve the desired therapeutic effect.

### Capsular Contracture

Capsular contracture is an example of a fibrous capsule formation and an undesired reaction following breast implant surgery and one of the reasons for reoperation following a breast implant surgery. Capsular contracture develops when a fibrous capsule or internal scar tissue forms a tight or constricting capsule around the implant to create a physical barrier between the foreign object and the rest of the body. As a result, the breast may feel painful and stiff, and the capsule may affect the appearance or shape of the breast. This complication most commonly requires invasive intervention, where the fibrous capsules and breast implants must be surgically removed.

Mast cells within the capsular tissue have been found to contain signaling protein tumor growth factor (TGF) b1. In addition to TGFb1, other signaling proteins associated with capsule contracture include tumor necrosis factor TNFa, matrix metalloproteinase MMP-2, tissue inhibitors of metalloproteinases (TIMP) 1 and TIMP-2. The presence of TNFa and MMP-2 in particular are associated with increased grades of capsular contractures, as graded on the Baker scale for capsular contractures.

Attempts to prevent capsular contracture include antibiotic irrigation, however these attempts have not had much success in reducing the occurrence of capsular contractures. Traditionally the capsular contractures are treated surgically, however this increases the risk of the reoccurrence of the contracture. Thus, there is an unmet need for safe and efficacious treatments for capsular contractures during breast implant surgeries.

The compositions herein may be used to treat capsular contractures. The compositions herein can be used, for example, to coat the surface of the implant, be co-administered prior to, with, or after the implantation of the implant, or be embedded within the implant, and can be configured to release over time (provide a controlled or delayed release) to achieve the desired therapeutic effect, for example, a reduction of about 20% to 100% of average capsule thickness compared to non-use of such fucan compositions, or a reduction of about 60 microns to 230 microns of average capsule thickness compared to non-use of such compositions. Use of the fucan compositions herein can also provide a reduction of about 20% to 100% of maximum capsule thickness compared to non-use of such compositions or a reduction of 100 microns to 1000 microns of maximum capsule thickness compared to non-use of such compositions. Use of the fucan compositions herein can also provide for an average capsule thickness of no more than about 228 microns, about 200 microns or about 190 microns after implantation, or a maximum capsule thickness of no more than about 442 microns, about 450 microns or about 540 microns after implantation.

### Implantable and Patient-Contacting Medical Devices

The surfaces of implantable and patient-contacting medical devices often attract proteins, bacteria, cells and tissue, causing the formation of a biofilm layer on the medical device surface. Such biofilm layers can, in turn, result in infection. Biofilm infections pose clinical challenges due to resistances to immune defense mechanisms and antimicrobials. Most implantable and patient-contacting medical devices are susceptible to microbial colonization and infection, which can result in dysfunction of the device, serious illness or death.

Attempts to treat biofilm infections with traditional compounds that inhibit the growth of or kill microbial infections, for example antibiotics, have been met with limited success due to the prevalence of resistance development in biofilm infections. Removal of an infected implanted medical device is possible, but it is largely invasive and therefore harmful to, and possibly dangerous to, the patient. Moreover, removal of an implanted medical device often still requires long term antimicrobial suppressive therapy.

One strategy to reduce the formation of biofilms and consequently reduce the prevalence of biofilm infections is to make the surfaces of medical devices and implants anti-adhesive, however limited success has been found with such approaches. The compositions herein can, for example, be used to coat the surface of the implant, be co-administered prior to, with, or after the implant, or be embedded within the implant. The compositions, systems, etc., herein can be configured to release over time (provide a controlled or delayed release) to achieve the desired therapeutic effect.

### Transplant Conditions

The surfaces of transplantable organs, tissues, etc. (e.g., heart, kidneys, liver, lungs, pancreas, intestine, thymus, uterus, musculoskeletal grafts such as bones and tendons, corneae, skin, heart valves, nerves and veins), can often attract proteins, bacteria, cells, tissue, antibodies and other immunological responses, etc. Such response can, in turn, result in infection, fibrosis, fibrous adhesions and GVHD.

Attempts to treat transplant conditions with traditional compounds such as antibiotics, have been met with limited success due to the prevalence of resistance development in transplant conditions or the inapplicability of such compounds to some transplant conditions such as GVHD or fibrous adhesions. Removal of problematic transplant may be possible in some instances, but even if possible it is largely invasive and therefore harmful to, and possibly dangerous to, the patient. Moreover, removal of a transplanted medical device often still requires long term antimicrobial or immunosuppressive therapy.

One strategy to reduce the formation of biofilms and consequently reduce the prevalence of transplant conditions is to make the surfaces of transplants anti-adhesive, however limited success has been found with such approaches in the past. The fucan compositions herein can, for example, be used to coat the surface of the transplant, be co-administered prior to, with, or after the transplant, or be embedded within the transplant. The compositions, systems, etc., herein can be configured to release over time (provide a controlled or delayed release) to achieve the desired therapeutic effect.

### Medical Device, Combination, Biologic and Pharmaceutical Products

The discussion herein also provides medical devices, combination, biologic and pharmaceutical products, comprising compositions as discussed herein in a medical device, combination product, biologic or pharmaceutically acceptable container. The products can also include a notice associated with the container, typically in a form prescribed by a governing agency regulating the manufacture, use, or sale of medical devices, combination, and pharmaceuticals or biopharmaceuticals, whereby the notice is reflective of approval by the agency of the compositions, such as a notice that the medically-acceptable fucan has been approved for human or veterinary administration to treat, for example, the capsular contracture or fibrous growth around implants or transplants discussed herein. Instructions for the use of the fucan herein may also be included. Such instructions may include information relating to the dosing of a patient and the mode of administration.

The present application is further directed to methods of making the various elements of the medically-acceptable fucans, systems etc., discussed herein, including making the medically-acceptable medical compositions themselves, as well as to methods of using the same, including for example treatment of the capsular contracture or fibrous growth around implants or transplants, diseases, etc., herein.

### Fucans

The medically-acceptable fucan compositions discussed herein may be modified to obtain medically-acceptable modified fucan compositions having low endotoxin levels. The medically-acceptable modified fucan compositions discussed herein may have an endotoxin level of less than about 0.2, 0.18, 0.12, 0.1, 0.09, 0.02, 0.01, 0.007, 0.005, 0.002 or 0.001 endotoxin units (EU) per milligram (mg) of the fucan (EU/mg).

The medically-acceptable fucans discussed herein may be modified to obtain medically-acceptable modified fucans having low total nitrogen levels by removing nitrogen containing compounds that may be attached to the medically-acceptable fucan. The medically-acceptable modified fucans, and compositions comprising the medically-acceptable modified fucans discussed herein may have a total nitrogen level of less than 0.2, 0.1, 0.08, 0.05, 0.03 or 0.02% w/w.

The medically-acceptable fucans and medically-acceptable modified fucans discussed herein may be incorporated in compositions further comprising any number of pharmaceutically acceptable excipients, for example, gelatin, hypromellose, lactose, water for injection USP, sodium chloride, sodium phosphate, sodium citrate, sodium ascorbate, phosphate buffers, citrate buffers, phosphate-citrate buffers, pluronic, cellulose, alginate, acrylate, hyaluronic acid, polyethylene glycol, poly(lactic acid), poly(lactic-co-glycolic acid), carrageenan, polyurethane, polyethylene, polypropylene, polytetrafluoroethylene, chitosan, injectable excipients and lactated Ringer's injection USP.

The medically-acceptable fucans and medically-acceptable modified fucans discussed herein may be administered in a composition comprising at least one of a paste, gel, patch, film, spray, liquid, lotion, cream, solution, suspension, solid, implant, microsphere, powder or other desired form. The medically-acceptable fucans and medically-acceptable modified fucans may be administered via intravenous, intraarticular, intralesional, intravaginal, rectal, intramuscular, intraperitoneal, subcutaneous, topical, intranasal, intraocular or oral administration routes. The medically-acceptable fucans and medically-acceptable modified fucans may be directly delivered to the site of the capsular contracture or fibrous growth around implants, or transplant conditions. The medically-acceptable fucans and medically-acceptable modified fucans may be continuously released to the site of the capsular contracture or fibrous growth around implants or transplants via controlled release from a polymeric or other acceptable controlled release dosage form. A solution or spray comprising the medically-acceptable fucans and/or medically-acceptable modified fucans herein may be used to irrigate, rinse, flush or wash the site of the capsular contracture or fibrous growth around implants, or transplant conditions The medically-acceptable fucans and medically-acceptable modified fucans may be applied as a coating on an implant or transplant that is delivered to a target site such as a surgical cavity.

The medically-acceptable fucans and medically-acceptable modified fucans discussed herein may be administered as a component of a pharmaceutical or biologic or combination product or medical composition comprising the medically-acceptable fucan/medically-acceptable modified fucan and at least one other drug. The drug may be at least one of paclitaxel, doxorubicin, camptothecin, etoposide, mitoxantrone, methotrexate, menadione, plumbagin, juglone, beta-laperchone cyclosporin, sulfasalazine, steroid, rapamycin, retinoid, docetaxel, colchicine, antisense oligonucleotide, ribozyme and vaccine.

The medically-acceptable fucans and medically-acceptable modified fucans discussed herein may be administered as a component of a pharmaceutical or biologic or combination product or medical composition comprising the medically-acceptable fucan/medically-acceptable modified fucan and at least one binder, adjuvant, excipient, etc.

The medically-acceptable fucan composition can be coated onto an implant, or transplant, including hydrophobic or hydrophilic implants, by a number of methods, for example: soaking the implant or transplant in or spraying the implant or transplant with the medically-acceptable fucan composition, by ionically, covalently or other binding of the medically acceptable fucan composition to the implant or transplant, by mixing the medically-acceptable fucan composition with the materials that are used to manufacture the implant or transplant prior to the manufacturing of the implant or transplant, and by ionically, covalently or other binding of the medically-acceptable fucan composition within the implant or transplant.

The medically-acceptable fucans and medically-acceptable modified fucans discussed herein may have a sulfation level of between about 10% w/w and 60% w/w, between about 20% w/w and 55% w/w, between about 30% w/w and 50% w/w, or between about 40% w/w and 45% w/w.

The medically-acceptable fucans and medically-acceptable modified fucans discussed herein may have a molar ratio of total fucose:total sulfate of between 1:0.5 and 1:4, between about 1:0.8 and 1:3.5, between about 1:1 and 1:2.5, between about 1:1.2 and 1:2.0, or between about 1:1.5 and 1:3.

The medically-acceptable fucans and medically-acceptable modified fucans discussed herein may have a molar ratio of total fucose and galactose:total sulfate of between about 1:0.5 and 1:4, between about 1:0.8 and 1:3.5, between about 1:1 and 1:2.5, between about 1:1.2 and 1:2.0, or between about 1:1.5 and 1:3.

The medically-acceptable fucans discussed herein may be modified to obtain medically-acceptable modified fucans having increased or decreased weight average molecular weight, number average molecular weight and/or peak molecular weight. The medically-acceptable fucans, having broad molecular weight distributions, discussed herein may be modified to obtain medically-acceptable modified fucans having a molecular weight distribution wherein a portion of the fucan at the low molecular weight end or at the high molecular weight end of the broad molecular weight distribution has been reduced or eliminated.

The molecular weight distribution of the medically-acceptable modified fucans may be measured using any desired, appropriate measurement system. Different systems can yield different readings or results from different compositions having essentially the same make-up, or even from the same batch when measured differently. One suitable measurement system is an aqueous gel permeation chromatography set up consisting essentially of one 300 mm analytical gel permeation chromatography column with a 7.8 mm inner diameter packed with hydroxylated polymethacrylate-based gel, having an effective molecular weight range of between about 50 kDa and about 5,000 kDa, one 300 mm analytical gel permeation chromatography column with a 7.8 mm inner diameter packed with hydroxylated polymethacrylate-based gel, having an effective molecular weight range of between about 1 kDa and about 6,000 kDa and one 40 mm guard column with a 6 mm inner diameter packed with hydroxylated polymethacrylate-based gel, the two analytical gel permeation chromatography columns and the one guard column contained in a column compartment at about 30° C., a refractive index detector at about 30° C., 0.1M sodium nitrate mobile phase run at 0.6 mL/min, and quantification against a peak molecular weight standard curve consisting essentially of a first dextran standard with a peak molecular weight of about 2,200 kDa, a second dextran standard with a peak molecular weight of between about 720 kDa and about 760 kDa, a third dextran standard with a peak molecular weight between about 470 kDa and about 510 kDa, a fourth dextran standard with a peak molecular weight between about 370 kDa and about 410 kDa, a fifth dextran standard with a peak molecular weight between about 180 kDa and about 220 kDa, and a sixth dextran standard with a peak molecular weight between about 40 kDa and 55 kDa. The peak molecular weight standard curve may further comprise a dextran standard with a peak molecular weight between 3 kDa and 5 kDa.

The medically-acceptable modified fucans discussed herein may have a weight average molecular weight greater than 300 kDa, for example between about 300 kDa and 2000 kDa, between about 350 kDa and 1500 kDa or between about 375 kDa and 1300 kDa.

The medically-acceptable modified fucans discussed herein may have a number average molecular weight greater than 100 kDa, for example between about 100 kDa and 800 kDa, between about 150 kDa and about 800 kDa or between about 170 kDa and 700 kDa.

The medically-acceptable modified fucans discussed herein may have a peak molecular weight greater than 200 kDa, for example between about 200 kDa and 800 kDa, between about 250 kDa and 750 kDa or between about 300 kDa and 700 kDa.

The medically-acceptable modified fucans discussed herein can have a molecular weight distribution wherein at least about 80% w/w or 90% w/w of the distribution is above 100 kDa. The medically-acceptable modified fucans discussed herein can have a molecular weight distribution wherein at least about 60% w/w, 70% w/w, 80% w/w or 90% w/w of the distribution is above 200 kDa. The medically-acceptable modified fucans discussed herein can have a molecular weight distribution wherein at least 20%, 40%, 50% or 70% of the distribution is above 500 kDa.

The medically-acceptable fucans and medically-acceptable modified fucans discussed herein may have a total carbohydrate content of between 27% w/w and 80% w/w, between about 30% w/w and 70% w/w, between about 40% w/w and 90% w/w, or between about 50% w/w and 100% w/w.

The medically-acceptable fucans and medically-acceptable modified fucans discussed herein may have a fucose content as a percentage of total carbohydrate of about 30% w/w and 100% w/w, between about 40% w/w and 95% w/w, or between about 50% w/w and 90% w/w.

The medically-acceptable fucans and medically-acceptable modified fucans discussed herein may have a galactose content as a percentage of total carbohydrate of about 0% w/w and 60% w/w, between about 3% w/w and 30% w/w, or between about 0% w/w and 10% w/w.

The medically-acceptable fucans and medically-acceptable modified fucans discussed herein may have a total glucuronic acid, mannose, rhamnose and xylose content as a percentage of the total carbohydrate content of less than about 30% w/w.

### EXAMPLES

Examples of methods that can be used to modify fucans and fucan compositions to obtain medically-acceptable modified fucans and medically-acceptable modified fucan compositions are discussed, for example, in the following publications, all of which are owned by the Owner/Applicant of the current application: PCT/CA2019/051025, filed Jul. 24, 2019; PCT/CA2019/051026, filed Jul. 24, 2019; PCT/CA2019/051027, filed Jul. 24, 2019, PCT/CA2019/051030, filed Jul. 24, 2019; PCT/CA2019/051028, filed Jul. 24, 2019; PCT/CA2020/050294, filed Mar. 5, 2020; PCT/CA2020/050295, filed Mar. 5, 2020; and PCT/CA2019/051029, filed Jul. 24, 2019. These methods can be applied to obtain medically-acceptable modified fucan compositions having low endotoxin levels, and/or other features as desired. For example, the medically-acceptable modified fucan compositions can have low levels of nitrogen-containing compounds, which could have interfered with the immunoassays discussed in this Examples section, as well as in patient treatments and other methods discussed herein.

### Example 1: Analysis of the Sulfate, Fucose, Galactose, Total Nitrogen Content and Molecular Weight Distribution of Three Medically-Acceptable Modified Fucans

The three medically-acceptable modified fucans in the medically-acceptable modified fucan compositions used in the Examples below were analyzed by the following methods to determine their physiochemical properties.

The three medically-acceptable modified fucans, hereafter referred to as Modified fucan 1, Modified fucan 2 and Modified fucan 3, were dissolved in 72% w/w sulfuric acid at 40 mg/mL and incubated at 45° C. in a water bath for 30 minutes. The resulting acid hydrolysates were then diluted to 4% w/w sulfuric acid in a high-pressure tube and incubated at 120° C. for 60 minutes. The resulting second acid hydrolysates were diluted to a 1/333 concentration with distilled water and run on a high performance anionic exchange column chromatography set up with pulsed amperometry detection (HPAE-PAD). Separation of analytes was accomplished by running 10 mM NaOH eluent at 1.0 mL/minute using an isocratic pump.

The fucose contents of the three medically-acceptable modified fucans were determined by interpolation on a standard curve for fucose. The galactose contents of the three medically-acceptable modified fucans were determined by the standard addition.

The medically-acceptable modified fucans were dissolved in deionized water, hydrolyzed under acidic conditions and analyzed by ICP-MS for % w/w total sulfur content. The sulfur content was converted to sulfate content by multiplying the sulfur content by the molar ratio of sulfate to sulfur to obtain % w/w sulfate content in the modified fucans.

The three medically acceptable modified fucans were analyzed for total nitrogen by igniting samples in a combustion analyzer and analyzing for nitrogen in the nitrous oxide gas produced using a thermal conductivity detector.

Gel permeation chromatography was used to evaluate the molecular weight distributions obtained for the three medically-acceptable modified fucans. There are a large number of different parameters, columns and standards available for use in gel permeation chromatography (GPC), resulting in a variety of instrumentation set-ups available for the analysis of molecular weight. For molecular weight determinations herein, the GPC were conducted using the following parameters: The mobile phase was 0.1M sodium nitrate run at 0.6 mL/min. The column compartment and detector were at 30° C. A Waters 2414 refractive index detector was used for detection.

Suitable GPC columns include GPC columns compatible with aqueous solvents, for example, columns packed with at least one of sulfonated styrene-divinylbenzene, NH-functionalized acrylate copolymer network, modified silica and hydroxylated polymethacrylate-based gel. For the analyses herein, three columns were used in series, comprising one 40 mm long guard column with an inner diameter (ID) of 6 mm packed with 6 μm particle size hydroxylated polymethacrylate-based gel, followed by a first 300 mm analytical GPC column with a 7.8 mm ID packed with 12 μm particle size hydroxylated polymethacrylate-based gel that has an exclusion limit of about 7,000 kDa and an effective molecular weight range of between about 50 kDa and about 5,000 kDa, followed by a second 300 mm analytical GPC column with a 7.8 mm ID packed with 10 μm particle size hydroxylated polymethacrylate-based gel that has an exclusion limit of about 7,000 kDa and an effective molecular weight range of between about 1 kDa and about 6,000 kDa. The total effective molecular weight range of the column set up was between about 1 kDa and about 6,000 kDa. An example of this column set up can be Ultrahydrogel® guard-Ultrahydrogel® 2000-Ultrahydrogel® Linear columns connected in series.

The samples of the three medically-acceptable modified fucans that were run were quantified against a standard curve containing traceable standards from the American Polymer Standards Corporation: DXT3755K (peak molecular weight=2164 kDa), DXT820K (peak molecular weight=745 kDa), DXT760K (peak molecular weight=621 kDa), DXT670K (peak molecular weight=401 kDa), DXT530K (peak molecular weight=490 kDa), DXT500K (peak molecular weight=390 kDa), DXT270K (peak molecular weight-196 kDa), DXT225K (peak molecular weight=213 kDa), DXT150K (peak molecular weight=124 kDa), DXT55K (peak molecular weight=50 kDa), DXT50K (peak molecular weight-44 kDa) and DXT5K (peak molecular weight=4 kDa), the peak molecular weights of these standards being between about 4 kDa and about 2,200 kDa. The standard curve used may, for example, include Dextran 3755 kDa, at least one of Dextran 50 kDa and Dextran 55 kDa, and between 3 to 6 additional traceable standards discussed herein, the calibration points being the peak molecular weights of the calibrants used. An example calibration curve may consist of DXT3755K, DXT 820K, DXT530K, DXT500K, DXT225K and DXT55K. The columns used herein had a total effective molecular weight range that encompassed and extended beyond the peak molecular weight range of the standards used for quantification of the three medically-acceptable modified fucans.

As is known, the identification of a molecular weight for a polymer including fucan/fucoidan, typically has a distribution of individual molecules having a variety of molecular weights. For a specified molecular weight identified within the distribution: there is a distribution of individual molecules of higher and lower molecular weights. The amount or percentage of individual polymers of a particular molecular weight increases or decreases as the molecular weight increases or decreases away from the specified molecular weight. The distribution may, but is not required to, have a generally Gaussian or distorted Gaussian shape.

The results for the total fucose, total galactose, total sulfate and molecular weight distribution of Modified fucan 1, Modified fucan 2 and Modified fucan 3 are presented in Table 1 below.

Results in the table below contain abbreviations used for certain characteristics of a molecular weight distribution. Peak molecular weight is denoted by PMW, weight average molecular weight is denoted by “WAMW”, number average molecular weight is denoted by “NAMW”, percentage distribution is denoted by “% dist”.

TABLE 1
Physiochemical properties of 3 different modified medically-acceptable fucans.

| | | | | % | % | % | | | | Total |
|---|---|---|---|---|---|---|---|---|---|---|
| | PMW | WAMW | NAMW | dist. >100 | dist. >200 | dist. >500 | Sulfate | Fucose | Galactose | Nitrogen |
| | (kDa) | (kDa) | (kDa) | kDa | kDa | kDa | (% w/w) | (% w/w) | (% w/w) | (% w/w) |
| Mod. | 686.1 | 1083.5 | 604.4 | 99.75 | 97.24 | 72.32 | 44.76 | 53.01 | 6.03 | <0.02 |
| fucan 1 | | | | | | | | | | |
| Mod. | 393.1 | 930.1 | 296.6 | 93.60 | 81.08 | 43.60 | 40.45 | 31.78 | 20.85 | <0.02 |
| fucan 2 | | | | | | | | | | |
| Mod. | 307.1 | 395.8 | 170.2 | 83.82 | 62.19 | 25.44 | 40.95 | 24.90 | 23.92 | 0.11 |
| fucan 3 | | | | | | | | | | |

“Mod.” = modified and medically-acceptable.

### Example 2: Endotoxin Measurement of Modified Fucan 1

Modified fucan 1 was tested for endotoxins using Associates of Cape Cod Pyrotell®-T lysate in accordance with the manufacturer's instructions. Turbidity measurements were taken using a Biotek Synergy® HTX incubating plate reader. Results were quantified against manufacturer CSE (control standard endotoxin) calibration curves. Modified fucan 1 was determined to have about 0.001 EU/mg.

### Example 3: In Vitro Immunoassay of Fucan Binding to Signaling Proteins

The binding of the three medically-acceptable modified fucans: Modified fucan 1, Modified fucan 2 and Modified fucan 3, to a variety of signaling proteins, including cytokines and chemokines, were studied herein via in vitro immunoassays to demonstrate the potential of fucans in inhibiting or sequestering these signaling proteins and consequently treating capsular contracture or fibrous growth around implants associated with the respective signaling proteins.

The following human antibody array immunoassay procedure was performed:

The three different medically-acceptable modified fucans discussed in Table 1 were dissolved in deionized water at 100 mg/mL, 10 mg/mL, 1 mg/mL and 0.1 mg/mL to provide fucan stock solutions. Each individual fucan stock solution was diluted 1:1 with Quantibody Sample Diluent (RayBiotech QA-SDB, RayBiotech, Georgia, USA). The fucan solution-diluent mixture was incubated at room temperature for about 1 hour. Each individual fucan solution-diluent mixture was then filtered through a 0.22 μm PVDF filter to obtain 50 mg/mL, 5 mg/mL, 0.5 mg/mL and 0.05 mg/mL fucan solution-diluent mixtures for each individual fucan.

A signaling protein standard mix containing numerous signaling protein standards was diluted in series to obtain a concentration series of signaling protein standards.

Each fucan solution-diluent mixture was further diluted 1:1 with the most concentrated signaling protein standard. A 1:1 mixture of deionized water and the most concentrated signaling protein standard was used as a blank. Each resulting fucan solution-diluent-signaling protein standard mixture was incubated for 1 hour at room temperature.

A plurality of slides (Human Kiloplex Quantitative Proteomics Array, RayBiotech®, QAH-CAA-X00-1), each containing 40 different antibodies in wells ordered into microarrays, each antibody specific for one signaling protein in the signaling protein standard, were provided. The antibodies were blocked with Quantibody® Sample Diluent (RayBiotech®, QA-SDB) by incubation for 30 minutes followed by decanting. 95 μL of each fucan solution-diluent-signaling protein standard mixture was then added to each of the wells. The mixtures were allowed to incubate in the wells for 2.5 hours. The same blocking and incubation procedures were followed for the concentration series of signaling protein standards. Post incubation, all samples and signaling protein standards were decanted from each well. The wells were washed with 150 μL 1× Wash Buffer 1, diluted from 20× Wash Buffer 1 (RayBiotech®, AA-WB1-30 ML) 5 times at room temperature, removing the wash solution from the wells after each wash. The wells were then washed with 150 μL 1× Wash Buffer 2, diluted from 20× Wash Buffer 2 (RayBiotech®, AA-WB2-30 ML) 2 times at room temperature, removing the wash solution from the wells after each wash.

80 μL of Quantibody® Sample Diluent (RayBiotech®, QA-SDB) containing a plurality of biotinylated antibodies, each specific for a signaling protein in the signaling protein standard, was added to each well and was left to incubate for between 1 and 2 hours. The solution was decanted from the wells and the wells again washed with 1× Wash Buffer 1 and 1× Wash Buffer 2 in the same manner.

80 μL of Quantibody® Sample Diluent (RayBiotech®, QA-SDB) containing Cy3 equivalent dye-conjugated streptavidin was added to each well and was left to incubate for about 1 hour in a dark room. The diluent solution was decanted from the wells and the wells were again washed with 150 μL 1× Wash Buffer 1 a total of 5 times, removing the wash solution from the wells after each wash.

The slide was further washed on a slide washer/dryer with 30 mL 1× Wash Buffer 1, then 30 mL 1× Wash Buffer 2, decanting the wash solution after each wash. Residual droplets were removed by drying with a N2 stream.

The slide was then imaged through the use of a laser scanner with a Cy3 wavelength. Results were obtained and transformed into signal reduction relative to the blank sample.

Table 2 below shows the immunoassay results, reported in percentage signal reduction compared to the blank for respective signaling proteins in the presence of the medically-acceptable modified fucans.

TABLE 2
Immunoassay results, reported in percentage signal reduction in the presence
of the medically-acceptable modified fucans, compared with the signal of
the blank, for signaling proteins. The 3 different medically-acceptable
modified fucans presented in this table are discussed in Example 1

| | Modified medically- | | | | Modified medically- | | | | Modified medically- | | | |
|---|---|---|---|---|---|---|---|---|---|---|---|---|
| | acceptable fucan 1 | | | | acceptable fucan 2 | | | | acceptable fucan 3 | | | |
| | Concentration (mg/mL) | | | | | | | | | | | |
| | 50 | 5 | 0.5 | 0.05 | 50 | 5 | 0.5 | 0.05 | 50 | 5 | 0.5 | 0.05 |
| IL-13 | 53% | 27% | 61% | 55% | 61% | 48% | 60% | 44% | 54% | 34% | 44% | 45% |
| IL-1b | 74% | 42% | 60% | 55% | 72% | 39% | 58% | 8% | 62% | 47% | 33% | 29% |
| IL-33 | 68% | 72% | 79% | 76% | 66% | 79% | 79% | 71% | 60% | 75% | 86% | 71% |
| MMP-2 | 44% | 24% | 38% | 35% | 73% | 47% | 38% | 40% | 34% | 17% | 35% | 56% |
| TGFb1 | 81% | 84% | 87% | 64% | 85% | 86% | 87% | 70% | 81% | 79% | 87% | 73% |
| TIMP-1 | 86% | 64% | 52% | 40% | 78% | 39% | 46% | 10% | 59% | 34% | 31% | 7% |
| TIMP-2 | 99% | 96% | 89% | 53% | 98% | 95% | 84% | 26% | 97% | 94% | 64% | 14% |
| TNFa | 50% | 21% | 28% | 35% | 53% | 36% | 37% | 28% | 38% | 1% | 8% | 24% |

The results in Table 2 demonstrate that the medically-acceptable modified fucans discussed herein inhibit a wide variety of signaling proteins, typically by significant and even highly significant amounts.

For certain signaling proteins, the medically-acceptable modified fucans discussed herein were able to inhibit or sequester at least about 50% of the signaling protein activity. For other signaling proteins, the medically-acceptable modified fucans were able to inhibit or sequester at least about 60% of the signaling protein activity. For yet other signaling proteins, the medically-acceptable modified fucans were able to inhibit at least about 70% of the signaling protein activity. For yet other signaling proteins, the medically-acceptable modified fucans were able to inhibit at least about 80% of the signaling proteins activity. For yet other signaling proteins, the medically-acceptable modified fucans were able to inhibit at least about 90% of the signaling protein activity. For yet other signaling proteins, the medically-acceptable modified fucans were able to inhibit at least about 95% of the signaling protein activity. For yet other signaling proteins, the medically-acceptable modified fucans were able to inhibit at least about 98% of the signaling protein activity.

The results in Table 2 also indicate that medically-acceptable modified fucans discussed herein, including the medically-acceptable modified fucans shown in Table 2, can inhibit or sequester a wide variety of signaling proteins at a target site in vivo, and therefore can be efficacious in the inhibition or treatment of the capsular contracture or fibrous growth around implants discussed herein.

Modified fucan 1, Modified fucan 2 and Modified fucan 3 were all able to inhibit or sequester between 64 and 87% TGFb1 activity, between 1 and 53% TNFa activity, between 17 and 73% MMP-2 activity, between 7 and 86% TIMP-1 activity and between 14 and 99% TIMP-2 activity. This indicates inhibition or sequestration of chemokines involved in the occurrence of capsular contractures by the medically acceptable modified fucans discussed herein.

Modified fucan 1, Modified fucan 2 and Modified fucan 3 were all able to inhibit or sequester between 64 and 87% TGFb1 activity, between 1 and 53% TNFa activity, between 8 and 74% IL-1b activity, between 27 and 61% of IL-13 activity, between and between 60 and 86% IL-33 activity. This indicates inhibition or sequestration of chemokines involved in the occurrence of FBR and fibrous capsule formation by the medically acceptable modified fucans discussed herein.

### Example 4: In Vivo Fibrous Capsule and Capsular Contracture Treatment with Fucoidan

Female New Zealand White rabbits of approximately 3 kg were premedicated with 22.5 mg/kg ketamine and 2.5 mg/kg xylazine. 5% isoflurane and oxygen were used for anesthetic induction and the rabbit was maintained on about 2% isoflurane and catheterized for IV infusion of LRS (3 mL/kg/h) for the remainder of the procedure. The back of the rabbit and the area around one hind limb was shaved. A Doppler cuff was placed on the hind leg shaved area and the animal was transferred to the surgical table.

A 3-cm transversal skin incision was made at the costa XII (13th rib) level in the middle lumbar-sacral region. A 3-cm long transversal incision was made in the panniculus carnosum (avoid cutting through muscle fascia). A tunnel was made by blunt dissection under the panniculus carnosum above the right rib cage to the right shoulder blade level. A 2.5 cm diameter pocket was made at the end of the tunnel. A method of capsule formation described in Table 3 below was applied to the pocket site. The fucoidan solution was applied to one pocket at a dose of <10 mL/kg body weight (about 4.5 mL of 3 mg/mL fucoidan solution), making sure to coat the entire pocket. For the control groups, no fucoidan solution was applied. A silicone implant (2 cm diameter×1 cm height) was inserted into the pocket. Before closing the incision, all bleeding was controlled. The panniculus carnosum incision was closed using 4-0 Vicryl in a continuous pattern. The subcuticular layer was sutured using 4-0 Monocryl. The skin incision layer was sutured using 4-0 Monocryl in a continuous subcuticular pattern. The same steps were repeated to the area above the left rib cage to the left of the shoulder blade level, the area to the right lumbar-sacral region at the right iliac crest level, and the area to the left lumbar-sacral region at the left iliac crest level for a total of 4 implants. Surgical glue was used after suturing if necessary.

Fibrous capsule thickness was quantitatively measured by selecting 4 different areas from a bisected capsule tissue section, and then within each of these areas taking 3 linear measurements perpendicular to the capsule surface. The average of these 3 measurements was recorded for each area by site. The average of the 4 areas was then again averaged to determine average capsule thickness per implant site, following which the average capsule thicknesses at the 4 sites were averaged to obtain an average capsule thickness per rabbit. The results of which are shown in Table 3 below.

TABLE 3
Average and maximum capsule thickness for fucoidan solution and
control groups using different methods of capsule formation

| Method | | | % reduction | | % reduction |
|---|---|---|---|---|---|
| of | Control or | Average | of average | Maximum | of maximum |
| capsule | fucoidan | capsule | capsule | capsule | capsule |
| forma- | solution | thickness | thickness | thickness | thickness |
| tion | group | (microns) | vs. control | (microns | vs. control |
| Desic- | Control | 288 | 21% | 702 | 23% |
| cation | Fucoidan | 228 | | 540 | |
| using | solution | | | | |
| O₂ gas | | | | | |
| stream | | | | | |
| Cautery | Control | 242 | 36% | 519 | 23% |
| | Fucoidan | 156 | | 399 | |
| | solution | | | | |
| TGFb1 | Control | 349 | 65% | 909 | 59% |
| | Fucoidan | 123 | | 374 | |
| | solution | | | | |
| TNFa | Control | 443 | 48% | 1042 | 49% |
| | Fucoidan | 229 | | 535 | |
| | solution | | | | |
| Leuko- | Control | 273 | 22% | 650 | 41% |
| triene | Fucoidan | 212 | | 381 | |
| B4 | solution | | | | |
| Lysed | Control | 374 | 48% | 1412 | 70% |
| whole | Fucoidan | 196 | | 425 | |
| rabbit | solution | | | | |
| blood | | | | | |

The results of the study demonstrate that fucoidan solution was effective in treating fibrous capsule and capsular contracture induced by various methods at the surgical site. Using fucoidan solution at the site of the implant resulted in between 21 to 65% reduction in average capsule thickness and between 23 to 70% reduction in maximum capsule thickness relative to untreated specimens.

### Example 14: In Vivo FBR and Fibrous Capsule Treatment with Fucoidan

Polyether-polyurethane sponge disc implants, 5 mm thick×12 mm diameter (Vitafoam Ltd, Manchester, U.K.) are used to induce a foreign body reaction within the peritoneal cavity. Four different treatment groups are used in the study as outlined in Table 4. The implants are sterilized, then either a) coated overnight by soaking the implants in a 50% w/v fucoidan solution; b) embedded with fucoidan by injecting a 50% w/v fucoidan solution into the implants; or c) left untreated, prior to the implantation surgery—for these untreated implants, in one group the implants are implanted without fucoidan to provide control implants, and in another group the implants are implanted along with the co-administration of fucoidan solution (0.05% w/v, 5 mL) co-administered via instillation with the implant in the peritoneal cavity. Male Wistar rats, weighing 300-350 g are anesthetized with a mixture of ketamine and xylazine (60 mg/kg and 10 mg/kg, respectively). The abdominal hair is shaved and the skin wiped with chlorohexidine and 70% ethanol. The implant is aseptically implanted inside the abdominal cavity through a 1 cm long ventral midline incision in the linea alba of the abdomen.

The incision is closed with silk braided nonabsorbable suture. At 10 days post-implantation, the animals are anesthetized with ketamine and xylazine and later euthanized by cervical dislocation. The implant is carefully dissected from adherent tissue, removed and weighed. They are then processed for histological staining, immunohistochemistry, and morphometric analysis.

The implants from both groups (control and treated) are fixed in 10% buffered formalin (pH 7.4) and processed for paraffin embedding. Sections with 5 mm thickness are stained with hematoxylin/eosin (H&E) and processed for light microscopic studies. Picrosirius-red staining followed by polarized-light microscopy are used to visualize and determine collagen fibers. Immunohistochemistry (IHC) reactions for the detection of endothelial cells/blood vessels are performed using the monoclonal antibody clone CD 31 (Fitzgerald MA, USA). Tissue sections (5 μm) are dewaxed and antigen retrieval is performed in citrate buffer (pH 6). The slides are boiled in citrate buffer for 25 min at 95° C. and then cooled for 1 h in the same buffer. Sections are incubated for 5 min in 3% hydrogen peroxide to quench the endogenous tissue peroxidase. Nonspecific binding is blocked by using normal goat serum for 10 min (1:10 in phosphate-buffered saline) with 1% bovine serum albumin (in phosphate-buffered saline). The sections are then immunostained with monoclonal antibody to CD31 (1:40 dilution, Dako Corporation, Carpinteria, CA, USA) for 60 min at room temperature then washed in Tris-HCl buffer. Sections are incubated for 30 min at room temperature with biotinylated Link Universal Streptavidin-HRP (Dako; Carpinteria, CA, USA). The reactions are revealed by applying 3,3′-diaminobenzidine in chromogen solution (DAB) (Dako; Carpinteria, CA, USA). The sections are counterstained with hematoxylin and mounted in Permount (Fisher Scientific; NJ, USA). Immunostaining is performed manually and the expression of proteins was evaluated on the basis of extent of cytoplasmic immunolabeling in endothelial cells forming lumen in six high-power fields, regardless of staining intensity (×400). The area of total collagen, capsule thickness and blood vessels are measured morphometrically to assess fibrous capsule formation.

To perform morphometric analysis of the number of blood vessels, images of cross sections are obtained from 35 fields and captured in light microscopy at 400× magnification. For collagen analysis and wall thickness, images are obtained from three representative fields at 200× magnification. The results of morphometric analysis are used to assess fibrous capsule thickness and fibrovascular tissue infiltration.

Rats that receive fucoidan-coated, fucoidan-embedded and fucoidan-co-administered implants have reduced fibrous capsule thickness and fibrovascular tissue infiltration than rats that receive just the implanted device without any fucoidan.

### Example 15: In Vivo FBR and Fibrous Capsule Treatment with Fucoidan

Cell implant systems, (for example, Cell Pouch System, Sernova, Corp., London, ON, Canada) are used to induce a foreign body reaction within the peritoneal cavity. Four different treatment groups are used in the study. The implants are sterilized, then either a) coated overnight by soaking the implants in a 50% w/v fucoidan solution; b) embedded with fucoidan by injecting a 50% w/v fucoidan solution into the implants; or c) left untreated, prior to the implantation surgery—for these untreated implants, in one group the implants are implanted without fucoidan to provide control implants, and in another group the implants are implanted along with the co-administration of fucoidan solution (0.05% w/v, 5 mL) co-administered via instillation with the implant in the peritoneal cavity. Male Wistar rats, weighing 300-350 g are anesthetized with a mixture of ketamine and xylazine (60 mg/kg and 10 mg/kg, respectively). The abdominal hair is shaved and the skin wiped with chlorohexidine and 70% ethanol. The implant is aseptically implanted inside the abdominal cavity through a 1 cm long ventral midline incision in the linea alba of the abdomen. The incision is closed with silk braided nonabsorbable suture. At 10 days post-implantation, the animals are anesthetized with ketamine and xylazine and later euthanized by cervical dislocation. The implant is carefully dissected from adherent tissue, removed and weighed. They are then processed for histological staining, immunohistochemistry, and morphometric analysis.

The implants from both groups (control and treated) are fixed in 10% buffered formalin (pH 7.4) and processed for paraffin embedding. Sections with 5 mm thickness are stained with hematoxylin/eosin (H&E) and processed for light microscopic studies. Picrosirius-red staining followed by polarized-light microscopy are used to visualize and determine collagen fibers. Immunohistochemistry (IHC) reactions for the detection of endothelial cells/blood vessels are performed using the monoclonal antibody clone CD 31 (Fitzgerald MA, USA). Tissue sections (5 μm) are dewaxed and antigen retrieval is performed in citrate buffer (pH 6). The slides are boiled in citrate buffer for 25 min at 95° C. and then cooled for 1 h in the same buffer. Sections are incubated for 5 min in 3% hydrogen peroxide to quench the endogenous tissue peroxidase. Nonspecific binding is blocked by using normal goat serum for 10 min (1:10 in phosphate-buffered saline) with 1% bovine serum albumin (in phosphate-buffered saline). The sections are then immunostained with monoclonal antibody to CD31 (1:40 dilution, Dako Corporation, Carpinteria, CA, USA) for 60 min at room temperature then washed in Tris-HCl buffer. Sections are incubated for 30 min at room temperature with biotinylated Link Universal Streptavidin-HRP (Dako; Carpinteria, CA, USA). The reactions are revealed by applying 3,3′-diaminobenzidine in chromogen solution (DAB) (Dako; Carpinteria, CA, USA). The sections are counterstained with hematoxylin and mounted in Permount (Fisher Scientific; NJ, USA). Immunostaining is performed manually and the expression of proteins was evaluated on the basis of extent of cytoplasmic immunolabeling in endothelial cells forming lumen in six high-power fields, regardless of staining intensity (×400). The area of total collagen, capsule thickness and blood vessels are measured morphometrically to assess fibrous capsule formation.

To perform morphometric analysis of the number of blood vessels, images of cross sections are obtained from 35 fields and captured in light microscopy at 400× magnification. For collagen analysis and wall thickness, images are obtained from three representative fields at 200× magnification. The results of morphometric analysis are used to assess fibrous capsule thickness and fibrovascular tissue infiltration.

Rats that receive fucoidan-coated, fucoidan-embedded and fucoidan-co-administered implants have reduced fibrous capsule thickness and less fibrovascular tissue infiltration compared to rats that receive just the implanted device without any fucoidan

### Example 16: In Vivo FBR Treatment with Fucoidan

Thirty mice are each surgically implanted with five silicone catheters intraperitoneally: an 8-mm midline incision is made in the abdomen, and five catheters are placed into the abdomen. Fifteen mice receive catheters coated with fucoidan solution (5 mg/mL) by submerging the catheter in the fucoidan solution for 4 hours prior to surgery. 15 mice receive uncoated catheters. Antibiotics (0.5 mg cefazolin and 1 mg gentamicin) are injected intraperitoneally just before closure. The muscle is closed with absorbable sutures, and the overlying skin is closed with wound clips. Catheters are recovered from 5 mice at 1, 3 and 5 weeks: the animals are anesthetized, and catheters and adherent cell layer are carefully removed from the abdomen.

Tissue samples of from the abdominal wall are imaged with trichrome staining to measure the thickness of the peritoneum submesothelial layer where the catheters were implanted. Mice that receive fucoidan coated catheters demonstrate between 50-90% thinner submesothelial layer thickness compared to mice with untreated catheters, indicating lower foreign body response.

### Example 17: In Vivo GVHD Treatment with Fucoidan

Ten pairs of donor matched mice 6-8 weeks old are sublethally irradiated with 2 Gy, anesthetized, and 1 mm fragments of human fetal thymus and liver are implanted under the kidney capsule bilaterally. One mouse in each donor matched pair is subsequently administered 0.5 mL of fucoidan solution (2.5 mg/mL) prior to closure and the other mouse of the pair is not given any further treatment prior to closure. Additionally, CD34+ cells are isolated from fetal liver via anti-CD34 microbeads and 1×10⁵ cells are injected intravenously within 6 hours post surgery.

Histologically, skin involvement in GVHD is characterized by a lymphocytic infiltrate of the epidermis, hair follicle, and dermal/subcutaneous junction with corresponding dropout of hair follicles, loss of subcutaneous fat, epidermal hyperplasia and hyalinization of dermal collagen, features similar to those observed in human GVHD or scleroderma. GVHD in pairs of donor matched mice is characterized by blinded scoring (0-4) of four components of skin histology (inflammation, epidermal hyperplasia, fibrosis and subcutaneous lipoatrophy) conducted over 40 weeks. Mice that receive fucoidan solution prior to closure demonstrate between 50-90% lower inflammation, epidermal hyperplasia, fibrosis and subcutaneous lipoatrophy scores than mice that do not receive fucoidan solution.

### Example 18: In Vivo Fibrosis and Transplant Fibrous Adhesions Treatment with Fucoidan

Ten pairs of mice 25-30 g are used in this study, half of them are donors and the other half recipients.

Donor Transplant Operation: The abdomen is entered via a long midline incision. The left kidney is exposed by moving the intestine laterally to the right side and using a mosquito clamp to retract the stomach. The left kidney is isolated by ligating and dividing the adrenal and testicular vessels with 8-0 silk sutures. After ligating and dividing lumbar branches, the aorta and inferior vena cava (IVC) are mobilized at their junction with the left renal artery and vein. Four sutures are tied around the aorta and IVC above and below the renal artery and vein. The left ureter is dissected free from the renal hilus to the bladder; the distal 3-5 mm of donor ureter is cleared of surrounding fat tissue. The aorta is ligated above the renal artery, a 30-gauge needle is introduced into the infrarenal aorta, and the graft is slowly perfused in situ with 0.5-1 ml of cold, heparinized Ringer's lactate solution (heparin concentration=100 U/ml). The renal vein is transected at its junction with the IVC. The aorta is divided obliquely, approximately 2 mm below the renal artery. The kidney and its vascular supply, along with the ureter attached is removed en bloc and stored in Ringer's lactate solution (n=5 with and n=5 without 2.5 mg/mL fucoidan solution) at 4° C.

Recipient Transplant Operation: A midline incision is made, the intestine is covered with wet gauze and retracted to the left side. The recipient's native right kidney is removed first. After ligating the lumbar branches, the infrarenal aorta and IVC are carefully isolated and cross-clamped with two 4 mm microvascular clamps. Retracting with an 11-0 nylon suture through a full thickness of aorta, an elliptical aortotomy (approximately one-fifth the diameter of vessel) is made with a single cut using iris scissors. A longitudinal venotomy (0.18 mm) is made in the IVC by first puncturing it with a 30-gauge needle and then snipping with iris scissors at a slightly lower level than the aortotomy (0.08 mm). Both the aorta and IVC are flushed thoroughly with heparinized saline. Two stay sutures are placed at both apices of the venotomy. The donor kidney is then removed from the ice, and placed in the right flank of the mouse. An end-to-side anastomosis between donor renal vein and recipient IVC is performed using continuous 10-0 sutures. The posterior wall is sutured within the vessel lumen without repositioning the graft. The anterior wall is then closed externally using the same suture. Once the venous anastomosis is completed the sutures are tied. An arterial anastomosis is performed in the same fashion as the venous anastomosis described herein. Gentle pressure is applied to the anastomotic site with a dry cotton swab for 1-2 minutes after revascularization. Two small holes are made by piercing through the lateral walls of the bladder using a 25-gauge needle or a pair of microsurgical forceps. The end of the ureter is grasped and pulled through both holes. The donor ureter is fixed proximally to the exterior wall of the bladder by three stitches through the periuretal connective tissue using 10-0 nylon. The distal end of the ureter is severed and the remaining 1 mm of ureter is allowed to retract into the bladder cavity. The second bladder hole is closed and secured with stitches using a 10-0 suture. Prior to closing the renal cavity, mice that receive fucoidan soaked donor kidney transplants are administered an additional 1 mL of 5 mg/mL fucoidan in LRS, the other group receive 1 mL of LRS at the surgical site.

After 4 weeks, the mice are euthanized and adhesions in the renal cavity are scored (0-4) for strength and quantity. Mice that receive transplanted kidneys stored in fucoidan-LRS solution prior to surgery and that receive 5 mg/mL fucoidan solution at the surgical site prior to closing demonstrated between 70-90% fewer transplant adhesions that were 50-90% weaker in strength compared to mice that receive untreated transplanted kidneys and LRS at the surgical site prior to closing.

All terms used herein are used in accordance with their ordinary meanings unless the context or definition clearly indicates otherwise. Also unless expressly indicated otherwise, in this disclosure the use of “or” includes “and” and vice-versa. Non-limiting terms are not to be construed as limiting unless expressly stated, or the context clearly indicates, otherwise (for example, “including,” “having,” and “comprising” typically indicate “including without limitation”). Singular forms, including in the claims, such as “a,” “an,” and “the” include the plural reference unless expressly stated, or the context clearly indicates otherwise.

Unless otherwise stated, adjectives herein such as “substantially” and “about” that modify a condition or relationship characteristic of a feature or features of an embodiment, indicate that the condition or characteristic is defined to within tolerances that are acceptable for operation of the embodiment for an application for which it is intended.

The scope of the present methods, compositions, systems, etc., includes both means plus function and step plus function concepts. However, the claims are not to be interpreted as indicating a “means plus function” relationship unless the word “means” is specifically recited in a claim, and are to be interpreted as indicating a “means plus function” relationship where the word “means” is specifically recited in a claim. Similarly, the claims are not to be interpreted as indicating a “step plus function” relationship unless the word “step” is specifically recited in a claim, and are to be interpreted as indicating a “step plus function” relationship where the word “step” is specifically recited in a claim.

From the foregoing, it will be appreciated that, although specific embodiments have been discussed herein for purposes of illustration, various modifications may be made without deviating from the spirit and scope of the discussion herein. Accordingly, the systems and methods, etc., include such modifications as well as all permutations and combinations of the subject matter set forth herein and are not limited except as by the appended claims or other claim having adequate support in the discussion and figures herein.

What is claimed is:

1. A method of treating a fibrous capsule formation in a patient at a site of an implant on or within the patient, the method comprising treating the fibrous capsule formation with a therapeutically effective amount of a medically acceptable fucan composition.

2. A method of treating a foreign body response in a patient at a site of an implant on or within the patient, the method comprising treating the foreign body response with a therapeutically effective amount of a medically acceptable fucan composition.

3. A method of treating a capsular contracture in a patient at a site of an implant on or within the patient, the method comprising treating the capsular contracture with a therapeutically effective amount of a medically acceptable fucan composition.

4. A method of treating a biofilm infection in a patient at a site of an implant on or within the patient, the method comprising treating the biofilm formation with a therapeutically effective amount of a medically acceptable fucan composition.

5. The method of any of claims 1 to 4 wherein the treating comprises inhibiting.

6. The method of any of claims 1 to 5 wherein the implant comprises at least one of a medical device, drug or combination product.

7. The method of any of claims 1 to 6 wherein the implant is composed of at least one of a non-synthetic, biologic, naturally derived and synthetic material.

8. The method of any of claims 1 to 7 wherein the implant comprises at least one of a bone plate, fracture fixation device, hip prostheses, knee prostheses, shoulder prostheses, ankle prostheses, elbow prostheses, artificial ligament, artificial tendon, cellular therapy, gene therapy, pacemaker encapsulation, catheter, stent, artificial heart valve, artificial artery, drug reservoir device for sustained release, diabetes monitor, insulin pump, skin repair device, breast implant, cochlear replacement, ocular lens, vascular graft, nerve conduit, surgical mesh, organ, tissue and cell.

9. The method of any one of claims 1 to 8 wherein the implant is composed of at least one of: autograft, allograft, fibrin, poly(lactic acid), poly(lactic-co-glycolic acid), alginate, carrageenan, hyaluronan, heparin, synthetic polyurethane, polyester, silicone, aluminium, steel, titanium, cobalt, chromium, nickel, gold, silver, platinum, metal alloy, calcium phosphate, hydroxyapatite, inorganic salt derivatives, alumina, zirconia, bioactive glass, porcelain, carbon, cyclic olefin copolymer, polycarbonate, polyetherimide, polyvinylchloride, polyethersulfone, polyethylene, polytetrafluoroethylene, polyetheretherketone, polypropylene, silicone, hydrogel, cellulose, starch, protein, peptide, DNA, RNA, collagen, gelatin, silk, chitin, chitosan, glucose, heart valve, blood vessel and liver tissue.

10. The method of any one of claims 1 to 9 wherein the treating with the therapeutically effective amount of the medically acceptable fucan composition comprises coating at least one surface of the implant with the therapeutically effective amount of the medically acceptable fucan composition before delivering the implant to the patient.

11. The method of any one of claims 1 to 9 wherein the treating with the therapeutically effective amount of the medically acceptable fucan composition comprises coating at least one surface of the implant with the therapeutically effective amount of the medically acceptable fucan composition after delivering the implant to the patient.

12. The method of any one of claims 1 to 9 wherein the treating with the therapeutically effective amount of the medically acceptable fucan composition comprises embedding the therapeutically effective amount of the medically acceptable fucan composition within the implant before delivering the implant to the patient.

13. The method of any one of claims 1 to 9 wherein the treating with the therapeutically effective amount of the medically acceptable fucan composition comprises embedding the therapeutically effective amount of the medically acceptable fucan composition within the implant after delivering the implant to the patient.

14. The method of any one of claims 1 to 9 wherein the treating with the therapeutically effective amount of the medically acceptable fucan composition comprises co-administering the therapeutically effective amount of the medically acceptable fucan composition with the implant.

15. The method of any one of claims 1 to 9 wherein the treating with the therapeutically effective amount of the medically acceptable fucan composition comprises administering the therapeutically effective amount of the medically acceptable fucan composition before delivering the implant to the patient.

16. The method of any one of claims 1 to 9 wherein the treating with the therapeutically effective amount of the medically acceptable fucan composition comprises administering the therapeutically effective amount of the medically acceptable fucan composition after delivering the implant to the patient.

17. The method of any of claims 14 to 16 wherein the administering occurs at the site of the implant.

18. The method of any of claims 1 to 17 wherein the patient is an animal.

19. The method of any of claims 1 to 18 wherein the medically acceptable fucan composition comprises at least one of a paste, gel, patch, film, spray, liquid, lotion, cream, solution, suspension, solid, implant, powder and microsphere.

20. The method of any of claims 1 to 19 wherein the medically acceptable fucan composition further comprises at least one medically acceptable excipient selected from the group consisting of gelatin, hypromellose, lactose, water for injection USP, sodium chloride, sodium phosphate, sodium citrate, sodium ascorbate, phosphate buffers, citrate buffers, phosphate-citrate buffers, pluronic, cellulose, alginate, acrylate, hyaluronic acid, polyethylene glycol, poly(lactic acid), poly(lactic-co-glycolic acid), alginate, carrageenan, polyurethane, polyethylene, polypropylene, polytetrafluoroethylene, chitosan and lactated Ringer's injection USP.

21. The method of any of claims 1 to 20 wherein the medically acceptable fucan composition comprises less than about 200 Endotoxin Units (EU).

22. The method of any of claims 1 to 20 wherein the medically acceptable fucan composition comprises less than about 100 Endotoxin Units (EU).

23. The method of any of claims 1 to 20 wherein the medically acceptable fucan composition comprises less than about 50 Endotoxin Units (EU).

24. The method of any of claims 1 to 20 wherein the medically acceptable fucan composition comprises less than about 20 Endotoxin Units (EU).

25. The method of any of claims 1 to 20 wherein the medically acceptable fucan composition comprises less than about 10 Endotoxin Units (EU).

26. The method of any of claims 1 to 20 wherein the medically acceptable fucan composition comprises less than about 5 Endotoxin Units (EU).

27. The method of any of claims 1 to 20 wherein the medically acceptable fucan composition comprises less than about 2 Endotoxin Units (EU).

28. Use of a medically acceptable fucan composition to treat fibrous capsule formation.

29. Use of a medically acceptable fucan composition to treat foreign body response.

30. Use of a medically acceptable fucan composition to treat capsular contracture.

31. Use of a medically acceptable fucan composition to treat biofilm infection.

32. A composition comprising an implant and a medically acceptable fucan composition.

33. The composition of claim 32 wherein the implant comprises at least one of a medical device, drug and combination product.

34. The composition of any of claim 32 or 33 wherein the implant is composed of at least one of a non-synthetic, biologic, naturally derived and synthetic material.

35. The composition of any of claims 32 to 34 wherein the implant comprises a bone plate, fracture fixation device, hip prostheses, knee prostheses, shoulder prostheses, ankle prostheses, elbow prostheses, artificial ligament, artificial tendon, cellular therapy, gene therapy, pacemaker encapsulation, catheter, stent, artificial heart valve, artificial artery, drug reservoir device for sustained release, diabetes monitor, insulin pump, skin repair device, breast implant, cochlear replacement, ocular lens, vascular graft, nerve conduit, surgical mesh, organ, tissue and cell.

36. The composition of any of claims 32 to 35 wherein the implant is composed of at least one of: autograft, allograft, fibrin, poly(lactic acid), poly(lactic-co-glycolic acid), alginate, carrageenan, hyaluronan, heparin, synthetic polyurethane, polyester, silicone, aluminium, steel, titanium, cobalt, chromium, nickel, gold, silver, platinum, metal alloy, calcium phosphate, hydroxyapatite, inorganic salt derivatives, alumina, zirconia, bioactive glass, porcelain, carbon, cyclic olefin copolymer, polycarbonate, polyetherimide, polyvinylchloride, polyethersulfone, polyethylene, polytetrafluoroethylene, polyetheretherketone, polypropylene, silicone, hydrogel, cellulose, starch, protein, peptide, DNA, RNA, collagen, gelatin, silk, chitin, chitosan, glucose, heart valve, blood vessel and liver tissue.

37. The composition of any of claims 32 to 36 wherein the composition comprises less than about 200 Endotoxin Units (EU).

38. The composition of any of claims 32 to 36 wherein the composition comprises less than about 100 Endotoxin Units (EU).

39. The composition of any of claims 32 to 36 wherein the composition comprises less than about 50 Endotoxin Units (EU).

40. The composition of any of claims 32 to 36 wherein the composition comprises less than about 20 Endotoxin Units (EU).

41. The composition of any of claims 32 to 36 wherein the composition comprises less than about 10 Endotoxin Units (EU).

42. The composition of any of claims 32 to 36 wherein the composition comprises less than about 5 Endotoxin Units (EU).

43. The composition of any of claims 32 to 36 wherein the composition comprises less than about 2 Endotoxin Units (EU).

44. A method of treating a transplant condition in a patient at a site of a transplant on or within the patient, the method comprising treating the transplant condition with a therapeutically effective amount of a medically acceptable fucan composition.

45. The method of claim 44 wherein the treating comprises inhibiting the transplant condition.

46. The method of any of claims 44 to 45 wherein the transplant comprises at least one of a heart, kidney, liver, lung, pancreas, intestine, thymus, uterus, bone and tendon, corneae, skin, heart valve, nerve or vein transplant.

47. The method of any one of claims 44 to 46 wherein the treating with the therapeutically effective amount of the medically acceptable fucan composition comprises coating at least one surface of the transplant with the therapeutically effective amount of the medically acceptable fucan composition before delivering the transplant to the patient.

48. The method of any one of claims 44 to 46 wherein the treating with the therapeutically effective amount of the medically acceptable fucan composition comprises coating at least one surface of the transplant with the therapeutically effective amount of the medically acceptable fucan composition after delivering the transplant to the patient.

49. The method of any one of claims 44 to 46 wherein the treating with the therapeutically effective amount of the medically acceptable fucan composition comprises embedding the therapeutically effective amount of the medically acceptable fucan composition within the transplant before delivering the transplant to the patient.

50. The method of any one of claims 44 to 46 wherein the treating with the therapeutically effective amount of the medically acceptable fucan composition comprises embedding the therapeutically effective amount of the medically acceptable fucan composition within the transplant after delivering the transplant to the patient.

51. The method of any one of claims 44 to 46 wherein the treating with the therapeutically effective amount of the medically acceptable fucan composition comprises co-administering the therapeutically effective amount of the medically acceptable fucan composition with the transplant.

52. The method of any one of claims 44 to 46 wherein the treating with the therapeutically effective amount of the medically acceptable fucan composition comprises administering the therapeutically effective amount of the medically acceptable fucan composition before delivering the transplant to the patient.

53. The method of any one of claims 44 to 46 wherein the treating with the therapeutically effective amount of the medically acceptable fucan composition comprises administering the therapeutically effective amount of the medically acceptable fucan composition after delivering the transplant to the patient.

54. The method of any of claims 44 to 53 wherein the administering occurs at the site of the transplant.

55. The method of any of claims 44 to 54 wherein the patient is an animal.

56. The method of any of claims 44 to 55 wherein the medically acceptable fucan composition comprises at least one of a paste, gel, patch, film, spray, liquid, lotion, cream, solution, suspension, solid, transplant, powder and microsphere.

57. The method of any of claims 44 to 56 wherein the medically acceptable fucan composition further comprises at least one medically acceptable excipient selected from the group consisting of gelatin, hypromellose, lactose, water for injection USP, sodium chloride, sodium phosphate, sodium citrate, sodium ascorbate, phosphate buffers, citrate buffers, phosphate-citrate buffers, pluronic, cellulose, alginate, acrylate, hyaluronic acid, polyethylene glycol, poly(lactic acid), poly(lactic-co-glycolic acid), alginate, carrageenan, polyurethane, polyethylene, polypropylene, polytetrafluoroethylene, chitosan and lactated Ringer's injection USP.

58. The method of any of claims 44 to 57 wherein the medically acceptable fucan composition comprises less than about 200 Endotoxin Units (EU).

59. The method of any of claims 44 to 57 wherein the medically acceptable fucan composition comprises less than about 100 Endotoxin Units (EU).

60. The method of any of claims 44 to 57 wherein the medically acceptable fucan composition comprises less than about 50 Endotoxin Units (EU).

61. The method of any of claims 44 to 57 wherein the medically acceptable fucan composition comprises less than about 20 Endotoxin Units (EU).

62. The method of any of claims 44 to 57 wherein the medically acceptable fucan composition comprises less than about 10 Endotoxin Units (EU).

63. The method of any of claims 44 to 57 wherein the medically acceptable fucan composition comprises less than about 5 Endotoxin Units (EU).

64. The method of any of claims 44 to 57 wherein the medically acceptable fucan composition comprises less than about 2 Endotoxin Units (EU).

65. Use of a medically acceptable fucan composition to treat transplant condition.

66. A composition comprising a transplant and a medically acceptable fucan composition adequate to treat a transplant condition.

67. The composition of any of claims 44 to 66 wherein the composition comprises less than about 200 Endotoxin Units (EU).

68. The composition of any of claims 44 to 66 wherein the composition comprises less than about 100 Endotoxin Units (EU).

69. The composition of any of claims 44 to 66 wherein the composition comprises less than about 50 Endotoxin Units (EU).

70. The composition of any of claims 44 to 66 wherein the composition comprises less than about 20 Endotoxin Units (EU).

71. The composition of any of claims 44 to 66 wherein the composition comprises less than about 10 Endotoxin Units (EU).

72. The composition of any of claims 44 to 66 wherein the composition comprises less than about 5 Endotoxin Units (EU).

73. The composition of any of claims 44 to 66 wherein the composition comprises less than about 2 Endotoxin Units (EU).
